(19) United States
(12) Patent Application Publication
(10) Pub. No.: US 2021/0369664 A1
(43) Pub. Date: Dec. 2, 2021

(54) PROPHYLACTIC OR THERAPEUTIC AGENT FOR PULMONARY HYPERTENSION WHICH COMPRISES PPAR? AGONIST

(71) Applicant: TOHOKU UNIVERSITY

(72) Inventors: Hiroaki SHIMOKAWA, Miyagi (JP); Kimio SATOH, Miyagi (JP); Taijyu SATOH, Yamagata (JP)

(73) Assignee: TOHOKU UNIVERSITY, Miyagi (JP)

(21) Appl. No.: 16/319,113

(22) PCT Filed: Jul. 20, 2017
(86) PCT No.: PCT/JP2017/026313

§ 371 Date: Jan. 18, 2019

(30) Foreign Application Priority Data
Jul. 20, 2016 (JP) 2016-141942

(51) Int. Cl. A61K 31/216; A61K 31/505; A61K 31/194; A61K 31/192; A61K 31/195; A61K 31/202; A61K 31/164; A61K 31/19; A61K 31/421; A61K 31/405; A61P 9/12

(52) U.S. Cl. CPC A61K 31/216; A61K 31/505; A61K 31/194; A61K 31/192; A61K 31/195; A61P 9/12; A61K 31/164; A61K 31/19; A61K 31/421; A61K 31/405; A61K 31/202

(57) ABSTRACT

A problem to be solved by the present invention is to provide a novel preventive or therapeutic agent for pulmonary hypertension containing as an active ingredient a compound that has not been known for a therapeutic effect on pulmonary hypertension heretofore. The present invention provides a preventive or therapeutic agent for pulmonary hypertension containing a PPARα agonist.

### TECHNICAL FIELD

The present invention relates to a preventive or therapeutic agent for pulmonary hypertension.

### BACKGROUND ART

Pulmonary hypertension is a disease involving increased blood pressure in pulmonary arteries, which carry blood from heart to lungs, leading to impaired cardiac and pulmonary functions, and is a disease quite different from a symptom generally called “hypertension”. In addition, pulmonary hypertension is a severe disease with high lethality, and hence there is an urgent need to develop a therapeutic method therefor.

Conventional treatments for pulmonary hypertension include vasodilation treatment using a catheter, and treatment such as surgical removal of thrombus, but less invasive therapeutic methods are desired. In addition, a vasodilator or the like is known as medication (e.g., Non-patent Literature 1), but there are still a large number of patients that cannot be saved by such therapeutic method. Thus, there is a strong demand for further development of a therapeutic agent for pulmonary hypertension.

### CITATION LIST

### Non-Patent Literature

NPL 1: J Clin Invest. 2012; 122(12): 4306-4313

NPL 2: Naunyn Schmiedebergs Arch Pharmacol. 2016 April; 389(4): 369-79

NPL 3: PLoS One. 2015 Jul. 24; 10(7): e0133391

NPL 4: The Journal of the Japanese Society of Internal Medicine Volume 103, No. 9: 2137-2143

### SUMMARY OF INVENTION

### Technical Problem

An object of the present invention is to provide a novel preventive or therapeutic agent for pulmonary hypertension containing as an active ingredient a compound that has not been known for a therapeutic effect on pulmonary hypertension heretofore.

### Solution to Problem

Under such circumstances, the inventors of the present invention have investigated thousands of kinds of compounds. As a result, the inventors have found that a compound having PPARα agonist activity suppresses excessive proliferation of pulmonary artery smooth muscle cells, which is supposed to be one of the causes for pulmonary hypertension, and has preventive and therapeutic effects on pulmonary hypertension. The present invention is based on such novel findings.

Thus, the present invention provides the following items:

Item 1. A preventive or therapeutic agent for pulmonary hypertension, including a PPARα agonist.

Item 2. The preventive or therapeutic agent for pulmonary hypertension according to Item 1, wherein the PPARα agonist is at least one kind selected from the group consisting of pirinixic acid, bezafibrate, clinofibrate, clofibrate, fenofibrate, ciprofibrate, 2-[[4-[2-[[(cyclohexylamino)carbonyl](4-cyclohexylbutyl) amino]ethyl]phenyl]thio]-2-methylpropanoic acid, leukotriene B4, oleylethanolamide, tetradecylthioacetic acid, N-((2S)-2-(((1Z)-1-methyl-3-oxo-3-(4-(trifluoromethyl)phenyl)prop-1-enyl)amino)-3-(4-(2-(5-methyl-2-phenyl-1,3-oxazol-4-yl) ethoxy)phenyl)propyl)propanamide, and 1-[(4-chlorophenyl) methyl]-3-[(1,1-dimethylethyl)thio]-α,α-dimethyl-5-(1-methylethyl)-1H-indole-2-propanoic acid, or a salt thereof.

Item 3. The preventive or therapeutic agent for pulmonary hypertension according to Item 2, wherein the PPARα agonist is at least one kind selected from the group consisting of pirinixic acid, bezafibrate, clofibrate, and fenofibrate, or a salt thereof.

Item 4. The preventive or therapeutic agent for pulmonary hypertension according to any one of Items 1 to 3, wherein the preventive or therapeutic agent for pulmonary hypertension is an orally administered agent.

Item 5-1. A method of preventing or treating pulmonary hypertension, including administering an effective dose of a PPARα agonist.

Item 5-2. The method according to Item 5-1, wherein the PPARα agonist is at least one kind selected from the group consisting of pirinixic acid, bezafibrate, clinofibrate, clofibrate, fenofibrate, ciprofibrate, 2-[[4-[2-[[(cyclohexylamino)carbonyl](4-cyclohexylbutyl) amino]ethyl]phenyl]thio]-2-methylpropanoic acid, leukotriene B4, oleylethanolamide, tetradecylthioacetic acid, N-((2S)-2-(((1Z)-1-methyl-3-oxo-3-(4-(trifluoromethyl)phenyl)prop-1-enyl)amino)-3-(4-(2-(5-methyl-2-phenyl-1,3-oxazol-4-yl) ethoxy)phenyl)propyl)propanamide, and 1-[(4-chlorophenyl) methyl]-3-[(1,1-dimethylethyl)thio]-α,α-dimethyl-5-(1-methylethyl)-1H-indole-2-propanoic acid, or a salt thereof.

Item 5-3. The method according to Item 5-2, wherein the PPARα agonist is at least one kind selected from the group consisting of pirinixic acid, bezafibrate, clofibrate, and fenofibrate, or a salt thereof.

Item 5-4. The method according to any one of Items 5-1 to 5-3, wherein the administering includes orally administering the PPARα agonist.

Item 6-1. A use of a PPARα agonist, for manufacture of a preventive or therapeutic agent for pulmonary hypertension.

Item 6-2. The use according to Item 6-1, wherein the PPARα agonist is at least one kind selected from the group consisting of pirinixic acid, bezafibrate, clinofibrate, clofibrate, fenofibrate, ciprofibrate, 2-[[4-[2-[[(cyclohexylamino)carbonyl](4-cyclohexylbutyl) amino]ethyl]phenyl]thio]-2-methylpropanoic acid, leukotriene B4, oleylethanolamide, tetradecylthioacetic acid, N-((2S)-2-(((1Z)-1-methyl-3-oxo-3-(4-(trifluoromethyl)phenyl)prop-1-enyl)amino)-3-(4-(2-(5-methyl-2-phenyl-1,3-oxazol-4-yl) ethoxy)phenyl)propyl)propanamide, and 1-[(4-chlorophenyl) methyl]-3-[(1,1-dimethylethyl)thio]-α,α-dimethyl-5-(1-methylethyl)-1H-indole-2-propanoic acid, or a salt thereof.

Item 6-3. The use according to Item 6-2, wherein the PPARα agonist is at least one kind selected from the group consisting of pirinixic acid, bezafibrate, clofibrate, and fenofibrate, or a salt thereof.

Item 6-4. The use according to any one of Items 6-1 to 6-3, wherein the preventive or therapeutic agent for pulmonary hypertension is an orally administered agent.

Item 7-1. A PPARα agonist, for use in prevention or treatment of pulmonary hypertension.

Item 7-2. The PPARα agonist according to Item 7-1, wherein the PPARα agonist is at least one kind selected from the group consisting of pirinixic acid, bezafibrate, clinofibrate, clofibrate, fenofibrate, ciprofibrate, 2-[[4-[2-[[(cyclohexylamino)carbonyl](4-cyclohexylbutyl) amino]ethyl]phenyl]thio]-2-methylpropanoic acid, leukotriene B4, oleylethanolamide, tetradecylthioacetic acid, N-((2S)-2-(((1Z)-1-methyl-3-oxo-3-(4-(trifluoromethyl)phenyl)prop-1-enyl)amino)-3-(4-(2-(5-methyl-2-phenyl-1,3-oxazol-4-yl) ethoxy)phenyl)propyl)propanamide, and 1-[(4-chlorophenyl) methyl]-3-[(1,1-dimethylethyl)thio]-α,α-dimethyl-5-(1-methylethyl)-1H-indole-2-propanoic acid, or a salt thereof.

Item 7-3. The PPARα agonist according to Item 7-2, wherein the PPARα agonist is at least one kind selected from the group consisting of pirinixic acid, bezafibrate, clofibrate, and fenofibrate, or a salt thereof.

Item 7-4. The PPARα agonist according to any one of Items 7-1 to 7-3, wherein the PPARα agonist is for use in prevention or treatment of pulmonary hypertension by oral administration.

### Advantageous Effects of Invention

According to the present invention, the novel preventive or therapeutic agent for pulmonary hypertension can be provided by using a PPARα agonist.

There is a previous report that pulmonary hypertension can be treated by the administration of a specific compound, i.e., pioglitazone or rosiglitazone (Non-patent Literatures 2 and 3). Each of pioglitazone and rosiglitazone is a compound having PPARγ agonist activity, and is not the PPARα agonist. PPARα and PPARγ have quite different in vivo functions. Specifically, it is known that PPARα has a function of, for example, reducing neutral fat in blood through its activation. PPARα is activated by a physiological ligand such as a free fatty acid or leukotriene B4, leading to a reduction in triglyceride concentration in blood through the proliferation of peroxisomes. Meanwhile, PPARγ is more closely related to glucose metabolism. PPARγ is expressed in a large number of tissues, such as heart, colon, kidney, pancreas, and spleen, and acts on the uptake of glucose in muscles. In addition, it is known that PPARγ has a function of, for example, increasing adiponectin to ameliorate insulin resistance in adipocytes. Accordingly, the effect of the present invention that pulmonary hypertension can be prevented or treated by using the PPARα agonist is unpredictable from the related art.

### BRIEF DESCRIPTION OF DRAWINGS

FIG. 1 are graphs for showing a right ventricular systolic pressure and a right ventricle/(left ventricle plus septum) weight ratio in Example 1.

FIG. 2 are graphs for showing the number of pulmonary vessels including thrombi per 100 mm² in a lung tissue slide (Pulmonary artery with clots) and a concentration ratio between D-dimer and TAT in a plasma sample (D-dimer/TAT ratio) in Example 1.

FIG. 3 are graphs for showing the results of Elastica-Masson staining of a lung tissue sample in Example 1. * means P<0.05.

FIG. 4 is a graph for showing the proliferation ratio of pulmonary artery smooth muscle cells in Example 2. * means P<0.05.

FIG. 5 are graphs for showing a right ventricular systolic pressure and a right ventricle/(left ventricle plus septum) weight ratio in Example 3. * means P<0.05.

### DESCRIPTION OF EMBODIMENTS

Preventive or Therapeutic Agent for Pulmonary Hypertension

The present invention provides a preventive or therapeutic agent for pulmonary hypertension, including a PPARα agonist.

As the PPARα agonist, a wide range of compounds known to have PPARα agonist activity in the field to which the present invention pertains may be used, and examples thereof include: pirinixic acid (WY-14643, CAS No. 50892-23-4, [[4-chloro-6-[(2,3-dimethylphenyl) amino]pyrimidin-2-yl]thio]acetic acid); bezafibrate (BEZATOL or Bezalip, CAS No. 41859-67-0, 2-[4-[2-[(4-chlorobenzoyl) amino]ethyl]phenoxy]-2-methylpropionic acid); clinofibrate (Lipoclin, CAS No. 30299-08-2, 2,2′-(4,4′-cyclohexylidenediphenoxy)-2,2′-dimethyldibutanoic acid); clofibrate (BINOGRAC, CAS No. 637-07-0, ethyl 2-(4-chlorophenoxy) isobutyrate); fenofibrate (Lipidil or Tricor, CAS NO. 49562-28-9, isopropyl 2-[4-(4-chlorobenzoyl)phenoxy]-2-methylpropionate); ciprofibrate (CAS No. 52214-84-3, 2-[4-(2,2-dichlorocyclopropyl) phenoxy]-2-methylpropionic acid); GW7647 (CAS No. 265129-71-3, 2-[[4-[2-[[(cyclohexylamino)carbonyl](4-cyclohexylbutyl) amino]ethyl]phenyl]thio]-2-methylpropanoic acid); leukotriene B4 (CAS No. 71160-24-2, 5S,12R-dihydroxy-6Z,8E,10E,14Z-eicosatetraenoic acid); oleylethanolamide (CAS No. 111-58-0, N-(2-hydroxyethyl)oleamide); tetradecylthioacetic acid (CAS No. 2921-20-2); GW6471 (CAS No. 880635-03-0, (N-((2S)-2-(((1Z)-1-methyl-3-oxo-3-(4-(trifluoromethyl)phenyl)prop-1-enyl)amino)-3-(4-(2-(5-methyl-2-phenyl-1,3-oxazol-4-yl)ethoxy)phenyl)propyl)propanamide))); and MK-886 (CAS No. 118414-82-7, 1-[(4-chlorophenyl)methyl]-3-[(1,1-dimethylethyl) thio]-α,α-dimethyl-5-(1-methylethyl)-1H-indole-2-propanoic acid)).

Of those PPARα agonists, preferred are pirinixic acid, bezafibrate, clofibrate, fenofibrate, salts thereof, and the like, and more preferred are: pirinixic acid represented by the following formula;

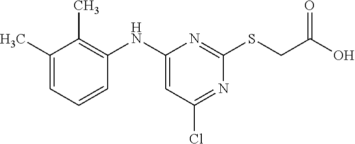 [SMILES: Cc1cccc(Nc2cc(Cl)nc(SCC(=O)O)n2)c1C]

fenofibrate represented by the following formula;

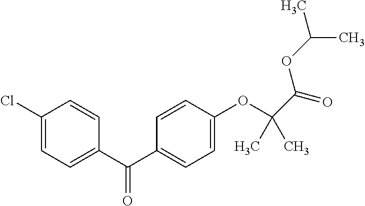 [SMILES: CC(C)OC(=O)C(C)(C)Oc1ccc(C(=O)c2ccc(Cl)cc2)cc1]

and salts thereof.

Those PPARα agonists may be used alone or in combination thereof. There is no previous report that the PPARα agonist has a preventive or therapeutic effect on pulmonary hypertension. Accordingly, the effect of the present invention is unpredictable from the related art.

In the present invention, examples of the PPARα agonist may include: low-molecular-weight compounds (e.g., compounds having molecular weights of 5,000 or less, preferably 2,000 or less, more preferably 1,000 or less, particularly preferably 500 or less), such as the compounds listed above; and high-molecular-weight compounds (compounds having molecular weights higher than those of the above-mentioned low-molecular-weight compounds, such as a compound having a molecular weight of more than 500, a compound having a molecular weight of more than 1,000, a compound having a molecular weight of more than 2,000, and a compound having a molecular weight of more than 5,000).

In addition, when the PPARα agonist or the salt thereof serving as the active ingredient of the present invention has an isomer, such as an optical isomer, a stereoisomer, or a regioisomer, the present invention may encompass both of an invention using any of the isomers and an invention using a mixture of a variety of isomers, unless it is clearly specified which of the isomers is used.

The salt of the PPARα agonist serving as the active ingredient of the present invention encompasses an acid addition salt and a salt with a base. Specific examples of the acid addition salt include: inorganic acid salts, such as a hydrochloride, a hydrobromide, a hydroiodide, a sulfate, a perchlorate, and a phosphate; organic acid salts, such as an oxalate, a malonate, a succinate, a maleate, a fumarate, a lactate, a malate, a citrate, a tartrate, a benzoate, a trifluoroacetate, an acetate, a methanesulfonate, a p-toluenesulfonate, and a trifluoromethanesulfonate; and acidic amino acid salts, such as a glutamate and an aspartate. Specific examples of the salt with a base include: alkali metal or alkaline earth metal salts, such as a sodium salt, a potassium salt, and a calcium salt; salts with organic bases, such as a pyridine salt and a triethylamine salt; and salts with basic amino acids, such as lysine and arginine. In addition, when the PPARα agonist serving as the active ingredient of the present invention is a cation, the salt of the PPARα agonist also encompasses a halide (e.g., a chloride) and the like.

The PPARα agonist serving as the active ingredient of the present invention may be present in the form of a hydrate or a solvate, and hence the compound serving as the active ingredient of the present invention also encompasses such hydrate and solvate.

A solvent forming the solvate is exemplified by alcohols, such as ethanol and propanol, organic acids, such as acetic acid, esters, such as ethyl acetate, ethers, such as tetrahydrofuran and diethyl ether, ketones, such as acetone, and dimethyl sulfoxide (DMSO).

In the present invention, the PPARα agonist or the salt thereof serving as the active ingredient of the present invention may be used alone as a preventive or therapeutic agent for pulmonary hypertension, or may be used as a pharmaceutical composition in combination with any of various pharmaceutically acceptable carriers (e.g., a tonicity agent, a chelating agent, a stabilizing agent, a pH regulator, a preservative, an antioxidant, a solubilizing agent, or a thickening agent).

Examples of the tonicity agent include: sugars, such as glucose, trehalose, lactose, fructose, mannitol, xylitol, and sorbitol; polyhydric alcohols, such as glycerol, polyethylene glycol, and propylene glycol; and inorganic salts, such as sodium chloride, potassium chloride, and calcium chloride.

Examples of the chelating agent include: edentates, such as disodium edetate, calcium disodium edetate, trisodium edetate, tetrasodium edetate, and calcium edetate; ethylenediaminetetraacetate; nitrilotriacetic acid or salts thereof; sodium hexametaphosphate; and citric acid.

An example of the stabilizing agent is sodium hydrogen sulfite.

Examples of the pH regulator include acids, such as hydrochloric acid, carbonic acid, acetic acid, and citric acid, and also include: alkali metal hydroxides, such as sodium hydroxide and potassium hydroxide; alkali metal carbonates or hydrogen carbonates, such as sodium carbonate; alkali metal acetates, such as sodium acetate; alkali metal citrates, such as sodium citrate; and bases, such as trometamol.

Examples of the preservative include: sorbic acid; potassium sorbate; parahydroxybenzoates, such as methyl parahydroxybenzoate, ethyl parahydroxybenzoate, propyl parahydroxybenzoate, and butyl parahydroxybenzoate; quaternary ammonium salts, such as chlorhexidine gluconate, benzalkonium chloride, benzethonium chloride, and cetylpyridinium chloride; alkylpolyaminoethylglycine; chlorobutanol; polyquad; polyhexamethylene biguanide; and chlorhexidine.

Examples of the antioxidant include sodium hydrogen sulfite, dried sodium sulfite, sodium pyrosulfite, and concentrated mixed tocopherols.

Examples of the solubilizing agent include sodium benzoate, glycerin, D-sorbitol, glucose, propylene glycol, hydroxypropyl methylcellulose, polyvinylpyrrolidone, macrogol, and D-mannitol.

Examples of the thickening agent include polyethylene glycol, methyl cellulose, ethyl cellulose, carmellose sodium, xanthan gum, sodium chondroitin sulfate, hydroxyethyl cellulose, hydroxypropyl cellulose, hydroxypropyl methylcellulose, polyvinylpyrrolidone, and polyvinyl alcohol.

In addition, the pharmaceutical composition may further contain, in addition to the PPARα agonist or the salt thereof, a compound known to have a preventive or therapeutic action on pulmonary hypertension. Examples of the compound known to have a preventive or therapeutic action on pulmonary hypertension include epoprostenol, sildenafil, and bosentan.

In the embodiment of the pharmaceutical composition, the content of the PPARα agonist or the salt thereof in the composition is not particularly limited, and may be appropriately set within, for example, conditions such as 90 mass % or more, 70 mass % or more, 50 mass % or more, 30 mass % or more, 10 mass % or more, 5 mass % or more, and 1 mass % or more in terms of the content of the PPARα agonist.

A dosage form is not particularly limited, and examples thereof may include various dosage forms including: orally administered agents, such as a tablet, a pill, a capsule, a powder, a granule, and a syrup; and parenterally administered agents, such as an injection (e.g., intravenous injection, intramuscular injection, or local injection), a gargle, a drop, external preparations (an ointment, a cream, a patch, and an inhalant), and a suppository. Of the dosage forms, for example, orally administered agents (e.g., a tablet, a pill, a capsule, a powder, a granule, and a syrup) and external preparations (an ointment, a cream, a patch, and an inhalant) are preferred.

In the present invention, the dose of the PPARα agonist or the salt thereof varies depending on, for example, an administration route and the age, body weight, or symptom of a patient, and hence cannot be uniquely defined. However, the dose only needs to be such an amount that a daily dose for adults is generally about 5,000 mg or less, preferably about 1,000 mg or less, more preferably 500 mg or less in terms of the dose of the PPARα agonist. The lower limit of the dose of the PPARα agonist or the salt thereof is also not particularly limited, and may be appropriately set within, for example, such a range that a daily dose for adults is generally 1 mg or more, preferably 10 mg or more, more preferably 100 mg or more in terms of the dose of the PPARα agonist. When administered once daily, the PPARα agonist or the salt thereof only needs to be contained in the above-mentioned amount in a single dose. When administered three times daily, the PPARα agonist or the salt thereof only needs to be contained in an amount corresponding to one-third of the above-mentioned amount in a single dose.

The preventive or therapeutic agent for pulmonary hypertension of the present invention is administered to patients, such as mammals. Examples of the mammals include humans, monkeys, mice, rats, rabbits, cats, dogs, pigs, cattle, horses, and sheep.

The preventive or therapeutic agent for pulmonary hypertension of the present invention prevents or treats pulmonary hypertension, in particular, pulmonary arterial hypertension, chronic thromboembolic pulmonary hypertension, or the like, by at least suppressing excessive proliferation of pulmonary artery smooth muscle cells. Accordingly, the present invention also provides a suppressor for excessive proliferation of pulmonary artery smooth muscle cells containing a PPARα agonist or a salt thereof. The active ingredient, dosage form, dose, and the like of the suppressor for excessive proliferation of pulmonary artery smooth muscle cells are the same as those of the preventive or therapeutic agent for pulmonary hypertension.

It is said that increased apoptosis resistance, mitochondrial dysfunction, and the like are involved in the excessive proliferation of pulmonary artery smooth muscle cells (Non-patent Literature 4). Therefore, even if a certain compound can suppress the proliferation of pulmonary artery smooth muscle cells without pulmonary hypertension, the compound cannot always suppress the excessive proliferation of pulmonary artery smooth muscle cells with pulmonary hypertension as well. That is, it can be said that the above-mentioned effect of the present invention has been found out for the first time by Examples and the like to be described later.

The present invention is more specifically described below by way of Examples. However, the present invention is not limited thereto.

### EXAMPLES

### Example 1

Hypoxia-induced pulmonary hypertension mice serving as a model generally used in an animal experiment for pulmonary hypertension were administered a PPARα agonist (WY-14643, Sigma) at 3 mg/kg/day by mixed feed. Specifically, first, 8-week-old male wild-type mice (Balb/c mice, n=12 per group) were housed in a transparent acrylic box with an oxygen concentration controlled to 10% using a hypoxia generator (Teijin Limited, Japan) under a 12 h light and dark cycle. The mice were stimulated by hypoxia for 3 weeks, and a diet in this period was mixed with WY-14643 (the following solution was used as WY-14643: 1 mg of WY-14643 was dissolved in 0.1 ml of DMSO, and the solution of WY-14643 in DMSO was mixed with ultrapure water (mQ or ultrapure water) to prepare a 100 ml solution). The administration by mixed feed was performed at 3 mg/kg/day according to the previous report (Andrew D, et al. Hepatology. 2012; 56: 281-290). A diet amount varied during the hypoxic stimulation, and hence the diet amount was measured every day to adjust a mixed feed concentration. The following tests were also performed as controls: a test in which the mice were stimulated by hypoxia in the same manner as described above except that the mice were administered a 1:1,000 (V/V) mixed solution of DMSO and ultrapure water by mixed feed in place of WY-14643 as a diet; and a test in which the mice were administered the mixed solution of DMSO and ultrapure water by mixed feed in place of WY-14643 as a diet and were not stimulated by hypoxia.

Assessment Method: Assessment of Pulmonary Hypertension

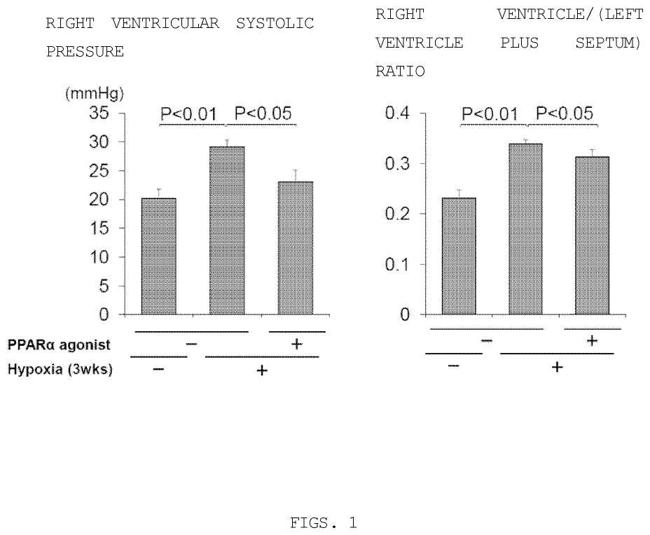

After the hypoxic stimulation for 3 weeks, a 1.2-Fr catheter (SciSense Inc., Ontario, Canada) was inserted in the jugular vein of the mice under isoflurane anesthesia and advanced into the right ventricle to measure a right ventricular systolic pressure. In addition, after the measurement of the right ventricular systolic pressure, the heart and the lungs were dissected. For the heart, the right ventricle and the left ventricle were separated and their weight ratio was measured to assess right ventricular hypertrophy. The results are shown in FIG. 1. The average body weights of the Control group (PPARα agonist: −, Hypoxia [3 wks]: −), the Vehicle group (PPARα agonist: −, Hypoxia [3 wks]: +), and the WY-14643 administration group (PPARα agonist: +, Hypoxia [3 wks]: +) before the test were 29.2±1.2 g, 28.9±1.0 g, and 29.6±1.4 g, respectively. In addition, the average body weights of the Control group, the Vehicle group, and the WY-14643 administration group after the test were 32.1±2.1 g, 23.5±1.8 g, and 25.7±2.0 g, respectively.

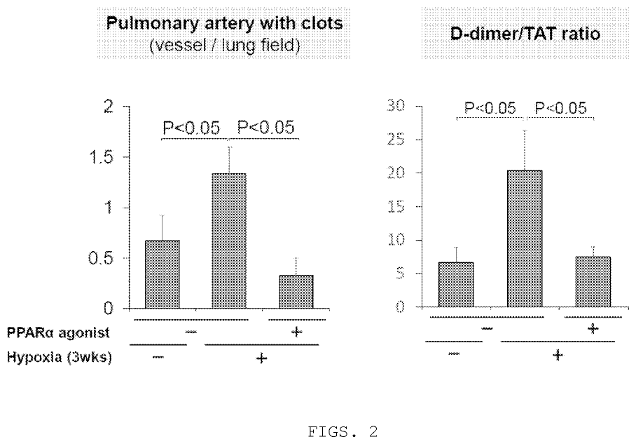

In addition, in tissue slides of the formalin-fixed lungs of each group, the number of pulmonary vessels including thrombi was counted for a field of about 100 mm², and the number of the pulmonary vessels including thrombi per 100 mm² was calculated. The results are shown in the “Pulmonary artery with clots” graph of FIG. 2. In addition, blood was collected using 3.8% citrate acid as an anticoagulant from the inferior vena cava of the mice of each group, and then centrifuged at 1,000 G for 15 minutes at 4° C. to obtain plasma. The resultant plasma sample was measured for its D-dimer concentration in plasma (unit: ng/ml) through the use of an ELISA kit (CEA506Mu manufactured by Life Science Inc.) according to the instruction of the kit. In addition, the plasma sample was measured for its thrombin-antithrombin complex (TAT) concentration in plasma (unit: pg/ml) through the use of an ELISA kit (SEA831Mu manufactured by Life Science Inc.) according to the instruction of the kit. A D-dimer/TAT concentration ratio calculated from the resultant numerical value is shown in the “D-dimer/TAT ratio” graph of FIG. 2.

The dissected lungs were fixed with 10% formalin for 24 hours, and then 3 μm sections of the specimens were used to prepare slides. The slides were subjected to Elastica-Masson staining for staining elastic fiber in order to assess vessel wall thickening (remodeling). 60 to 80 distal pulmonary arteries having diameters of from 20 μm to 70 μm were observed per mouse, and their wall thickening was divided into 3 stages. A case in which a double elastic lamina was visible for less than 50% of the entire periphery of the vessel was defined as mild, a case in which the double elastic lamina was visible for 50% or more and less than 100% of the entire periphery was defined as moderate, and a case in which the double elastic lamina was visible throughout the entire periphery was defined as severe.

Results and Discussion

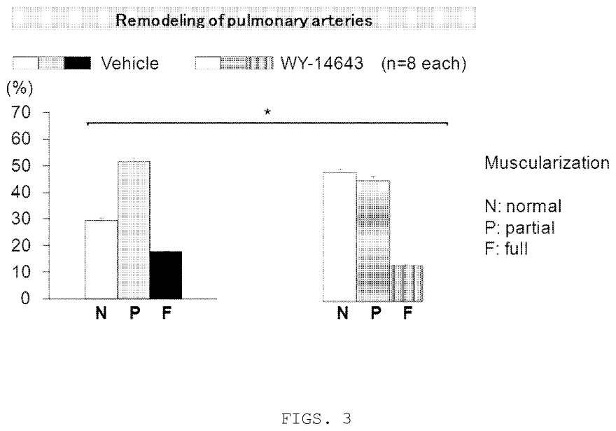

Significant reductions in right ventricular systolic pressure and right ventricular hypertrophy were found in the group administered WY-14643 by mixed feed (FIG. 1). In addition, the administration of WY-14643 by mixed feed suppressed the increase in thrombus formation found in the chronic hypoxic stimulation. In addition, the D-dimer/TAT ratio serving as an indicator of impaired fibrinolytic capacity was increased by the hypoxic stimulation, but its reduction was found in the group administered WY-14643 by mixed feed (FIG. 2). A significant reduction was also found in the remodeling of distal pulmonary vessels. That is, the result was that the administration of WY-14643 ameliorated pulmonary hypertension (FIG. 3).

### Example 2

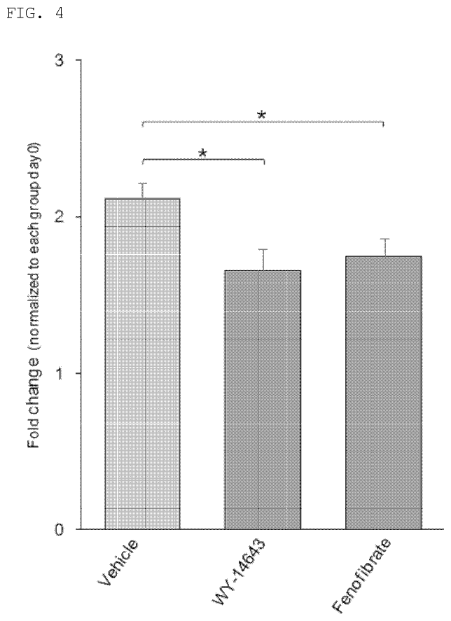

The effects of PPARα agonists WY-14643 and fenofibrate on the proliferation of pulmonary artery smooth muscle cells were assessed. Specifically, pulmonary artery smooth muscle cells were purchased from Lonza, and the cells were cultured. The cells were seeded in wells of a 96-well plate at 3,000 cells/well. On the next day, the number of the cells on day 1 was assessed by an MTT assay. On day 1, each of the PPARα agonists was added at 10 μM to the culture medium, and the cells were cultured for 3 days. On day 4, the MTT assay was performed again. The results are shown in FIG. 4. As shown in FIG. 4, the result was that cell proliferation was significantly suppressed in the fenofibrate group.

### Example 3

The influences of fenofibrate on the right ventricular systolic pressure (RVSP) and right ventricle/(left ventricle plus septum) ratio (RVH) of hypoxia-induced pulmonary hypertension mice were measured in the same manner as in Example 1 except that fenofibrate (the following solution was used as fenofibrate: fenofibrate was dissolved in DMSO, and the solution of fenofibrate in DMSO was mixed with ultrapure water (mQ or ultrapure water) to prepare a 100 ml solution, so as to achieve an intake of 50 mg/kg/day according to the previous report) was used in place of WY-14643. The average body weights of the Vehicle group and the fenofibrate administration group before the test were 184.89±1.25 g and 185.83±5.96 g, respectively. In addition, the average body weights of the Vehicle group and the fenofibrate administration group after the test were 318.75±17.80 g and 334.30±20.67 g, respectively.

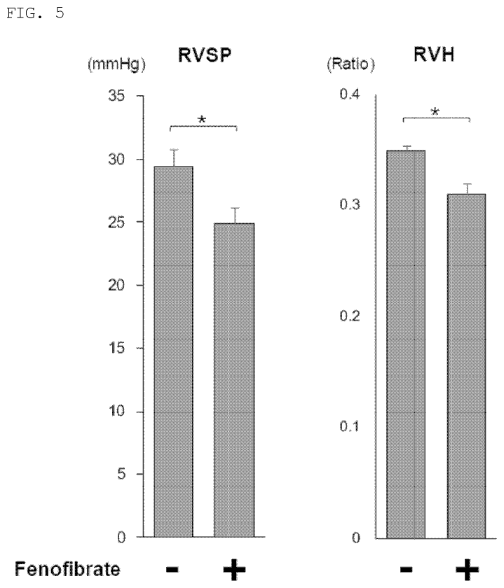

The results are shown in FIG. 5. As shown in FIG. 5, significant reductions in right ventricular systolic pressure and right ventricular hypertrophy were found in the group administered fenofibrate by mixed feed.

1-7. (canceled)

8. A method of preventing or treating pulmonary hypertension, comprising administering an effective dose of a PPARα agonist.

9. The method according to claim 8, wherein the PPARα agonist is at least one kind selected from the group consisting of pirinixic acid, bezafibrate, clinofibrate, clofibrate, fenofibrate, ciprofibrate, 2-[[4-[2-[[(cyclohexylamino)carbonyl](4-cyclohexylbutyl) amino]ethyl]phenyl]thio]-2-methylpropanoic acid, leukotriene B4, oleylethanolamide, tetradecylthioacetic acid, N-((2S)-2-(((1Z)-1-methyl-3-oxo-3-(4-(trifluoromethyl) phenyl)prop-1-enyl)amino)-3-(4-(2-(5-methyl-2-phenyl-1,3-oxazol-4-yl) ethoxy)phenyl)propyl)propanamide, and 1-[(4-chlorophenyl)methyl]-3-[(1,1-dimethylethyl) thio]-α,α-dimethyl-5-(1-methylethyl)-1H-indole-2-propanoic acid, or a salt thereof.

10. The method according to claim 9, wherein the PPARα agonist is at least one kind selected from the group consisting of pirinixic acid, bezafibrate, clofibrate, and fenofibrate, or a salt thereof.

11. The method according to claim 8, wherein an effective dose of the PPARα agonist is orally administered.

12. The method according to claim 9, wherein an effective dose of the PPARα agonist is orally administered.

13. The method according to claim 10, wherein an effective dose of the PPARα agonist is orally administered.

14. The method according to claim 8, wherein the pulmonary hypertension is pulmonary arterial hypertension.

15. The method according to claim 9, wherein the pulmonary hypertension is pulmonary arterial hypertension.

16. The method according to claim 10, wherein the pulmonary hypertension is pulmonary arterial hypertension.

17. The method according to claim 11, wherein the pulmonary hypertension is pulmonary arterial hypertension.

18. The method according to claim 12, wherein the pulmonary hypertension is pulmonary arterial hypertension.

19. The method according to claim 13, wherein the pulmonary hypertension is pulmonary arterial hypertension.
